(19) United States
(12) Patent Application Publication
(10) Pub. No.: US 2004/0077942 A1
(43) Pub. Date: Apr. 22, 2004

(54) 3D imaging for catheter interventions by use of positioning system

(76) Inventors: Andrew F. Hall, St. Charles, MO (US); John Rauch, St. Louis, MO (US); Joachim Hornegger, Baiersdorf (DE); Reinmar Killmann, Forchheim (DE); Norbert Rahn, Forchheim (DE); Johann Seissl, Erlanger (DE); Siegfried Wach, Hochstadt (DE); Benno Heigl, Untersiemau (DE)

Correspondence Address: Harness, Dickey & Pierce, P.L.C., Suite 400, 7700 Bonhomme, St. Louis, MO, 63105, US

(21) Appl. No.: 10/290,089

(22) Filed: Nov. 7, 2002

(30) Foreign Application Priority Data
Mar. 11, 2002 (DE) 10210645.2

(51) Int. Cl. A61B 5/05

(52) U.S. Cl. 600/428

(57) ABSTRACT

A method of locating and visualizing a medical catheter that has been introduced into an area of examination within a patient

A method of locating and visualizing a medical catheter that has been introduced into an area of examination within a patient, in particular during a cardiac examination or treatment, comprising the following steps:

using a 3D image data set of the area of examination and generating a 3D reconstructed image of the area of examination,

continuously or intermittently locating the spatial position of the tip of the catheter by means of a position locating system, with a position locating means being integrated into the tip of said catheter,

displaying the 3D reconstructed image and displaying the tip of the catheter in its accurate position in the 3D reconstructed image on a monitor, with the coordinate system of the position locating system and the coordinate system of the 3D reconstructed image being registered with respect to each other.

The subject matter of the present invention relates to a method of locating and visualizing a medical catheter that has been introduced into an area of examination within a patient, in particular during a cardiac examination or treatment.

Patients suffering from disorders are increasingly examined or treated by means of minimally invasive methods, i.e., methods that require the least possible surgical intervention. One example is treatments with endoscopes, laparoscopes, or catheters which are introduced into the area of examination inside the patient via a small opening in the body. Catheters are frequently used in cardiological examinations, for example, in the presence of cardiac arrhythmias which are today treated by means of so-called ablation procedures.

In such procedures, a catheter is introduced into a chamber of the heart under radiological guidance, i.e., by taking X-ray images via veins or arteries. In the cardiac chamber, the tissue that causes the arrhythmia is ablated by means of application of high-frequency electric current, which leaves the previously arrhythmogenic substrate behind in the form of necrotic tissue. The healing character of this method has significant advantages when compared to lifelong medication; in addition, this method is also economic in the long term.

From the medical and technical standpoint, the problem is that although during the intervention the catheter can be visualized very accurately and with high resolution in one or several X-ray images, which are also called fluoro images, the anatomy of the patient can only be inadequately visualized on the X-ray images. To track the catheter, generally two 2D X-ray images from two different directions of projection, in most cases orthogonal to each other, have so far been taken. Based on the information provided by these two images, the physician himself now has to determine the position of the catheter, something that is often accompanied by considerable uncertainty.

The problem to be solved by the present invention is to make available a possible visualization technique which makes it easier for the physician to observe the exact position of the catheter in the area of examination, i.e., for example, in the heart,

To solve this problem, a method of the type mentioned in the introduction using the following steps is made available:

using a 3D image data set of the area of examination and generating a 3D reconstructed image of the area of examination,

continuously or intermittently locating the spatial position of the tip of the catheter by means of a position locating system, with a position locating means being integrated into the tip of said catheter,

displaying the 3D reconstructed image and displaying the tip of the catheter in its accurate position in the 3D reconstructed image on a monitor, with the coordinate system of the position locating system and the coordinate system of the 3D reconstructed image being registered with respect to each other.

The method according to the present invention makes it possible during the examination to visualize the catheter—which is a flexible and bendable, i.e., nonrigid instrument—practically in real-time in a three-dimensional image of the area of examination, i.e., the heart or a central vascular tree of the heart, in the correct position in the volume image. This is made possible, on the one hand, by the fact that a three-dimensional reconstructed image of the area of examination is generated using a 3D image data set. On the other hand, the use of a catheter with a position locating means—which is integrated into the tip and the spatial coordinates of which can be located by means of a suitable external position locating system in a coordinate system which has its own position locating system—makes it possible to accurately identify the spatial position of the tip of the catheter when this tip is already present in the area of examination. Since the two coordinate systems of the 3D image data set and the 3D reconstructed image and the position locating system are registered with respect to each other, it is now possible, using a suitable transformation matrix, to transform the coordinates of the tip from the coordinate system of the position locating system into the coordinate system of the 3D reconstructed image so that the tip of the catheter can be positionally accurately visualized in the 3D volume image. As a result, the physician can very accurately visualize the tip of the catheter in its actual position in the area of examination, the relevant anatomical details of which he can also see very accurately and in high resolution from the 3D volume image. This makes possible an easy navigation of the catheter.

To register the two coordinate systems, a first alternative according to the invention provides that defined markings be used in the 3D reconstructed image and, correspondingly, in the coordinate system of the position locating system. This means that in both coordinate systems, the same markings are defined so that it is possible to register both coordinate systems with respect to each other, using a suitable transformation matrix which displays the markings so as to be superimposed over one another. The markings in the 3D reconstructed image can, for example, be interactively defined by the user via a mouse. The markings in the coordinate system of the position locating system can be defined, for example, by moving the catheter to the marking positions. It is possible, on the one hand, to use external markings as long as the corresponding markings can also be seen in the 3D reconstructed image. External markings that can be used are markings or similar identifications that are placed on the patient. It is also possible to use internal markings, with these markings, under simultaneous radiological guidance, being targeted and thus defined with the catheter that has been introduced into the area of examination. This means that the physician moves the catheter to specific points in the area of examination which have already been specified in the 3D reconstructed image, e.g., to certain vascular branching points or similar structures. Once he reaches such a point, this point can be defined as a marking. If the external markings mentioned earlier are used, the positions of these markings are defined by moving the catheter to the position of the marking.

The second alternative provides that visible markings that are place on the outside of the patient be used as markings in the 3D reconstructed image.

To ensure an accurate image registration, it suffices in most cases if at least three markings and preferably always the same number of markings are defined in each coordinate system, since a minimum of three pairs of markings ensures that the coordinate systems are accurately positioned with respect to each other is detected and are described by means of a transformation matrix.

After at least three pairs of markings have been identified, a 3D/3D registration is carried out. The result is a transformation matrix in which the translation, orientation, and scaling parameters are included. The transformation matrix describes the registration between the image coordinates and the coordinates of the positioning system so that during the subsequent catheter intervention or during the already concluded catheter intervention, the coordinates of the positioning system can be transformed into image coordinates. Altogether, the two registration alternatives described are marking- and/or landmark-based registrations.

In addition, it is possible to carry out a so-called “surface-based” registration. For this purpose, several points of the area of examination shown in the 3D reconstructed image can be defined by means of a segmentation algorithm and their coordinates can be located, with the catheter that has been introduced into the area of examination being moved to several points which are thereby defined and their coordinates located so that a specific area is defined by these points, and for image registration, the transformation matrix is calculated on the basis of the points, using a suitable surface matching algorithm. Using the positioning means in the catheter, in this embodiment of the invention, several points on the intracardiac surface of the heart which are targeted by the catheter are recorded. Together, these points create a netlike image of the surface of the heart in the area into which the catheter has been introduced; thus, for each point, the 3D position coordinates are stored and subsequently evaluated to describe the scanned surface. This can also be done under radiological guidance, which allows the physician to see which areas he has already scanned in this manner. In the 3D reconstructed image, corresponding points are located using a segmentation algorithm; this means that a surface area is defined there as well. Using a surface matching algorithm, a transformation matrix which matches the two surfaces to each other is subsequently calculated. Again, in the transformation matrix, the translation, rotation, and scaling parameters are included. To calculate the transformation matrix, known surface matching algorithms, such as the ICP algorithm (ICP=Iterative Closest Point) or a hierarchical chamfer matching algorithm, can be used.

A third possibility of image registration provides for the use of a sensor element of the position locating system on a C-shaped arm of an X-ray image-taking device which comprises an isocenter and with which the 3D image data set had been acquired, with the 3D reconstructed image being reconstructed relative to the isocenter. This is based on the assumption that the 3D image data set was acquired with a 3D angiography device in which the position and orientation of the 3D reconstructed image relative to the image-taking direction are known. Since a sensor element of the position locating system, above which the coordinate system of the position locating system is mounted, is also attached to the C-shaped arm of this image-taking device, this coordinate system is also defined relative to the isocenter. Thus, if the 3D reconstructed image is subsequently reconstructed relative to the isocenter of the C-shaped arm, its orientation and position are immediately known in the coordinate system of the position locating system as well. Thus, if the 3D image data set is acquired intraoperatively, which means shortly before the actual intervention is carried out, i.e., when the patient is already lying on the treatment table of the image-taking device, and if the patient does not move during the subsequent intervention, no separate registration is required. Only if the patient moves will it be necessary to acquire and reconstruct a new 3D image data set by means of the image-taking device. In any case, it is principally possible to continuously position and orient the tip of the catheter in the 3D reconstructed image during the intervention, without necessarily carrying out a registration prior to the intervention.

If the area of examination is an area which moves rhythmically or arrhythmically, for example, the heart, care must be taken to ensure that in order to visualize the area of examination accurately, the 3D reconstructed image and the acquired position data were taken in the same phase of motion. For this purpose, it can be provided that to reconstruct the 3D reconstructed image, only those image data which are acquired during a specific phase of motion that is acquired parallel to the acquisition of the image data be used, with the phase of motion also being acquired while the position of the catheter is being located and with the position data only being acquired when the area of examination is located in the same phase of motion in which the 3D reconstructed image is reconstructed. In this manner, it is ensured that the position of the catheter is determined in the same phase as the reconstructed volume image and that thus, the position of the tip of the catheter can be accurately determined and superimposed. Principally, two possibilities are available; first, the 3D reconstructed image can be reconstructed in a specific phase of motion which subsequently specifies the position data acquisition phase. Alternatively, it is also possible for the position data to be acquired in any phase of motion, in that case, however, always in the phase on which the reconstruction and the image data used therefore are based on. An example for the acquisition of the phase of motion is a simultaneously recorded ECG which records the movements of the heart. Based on the ECG, it is subsequently possible to choose the relevant image data. To acquire the position data, the position locating system can be triggered via the ECG, which ensures that the position data are always taken in the same phase of motion. Alternatively, it is also possible to record the respiratory phases of the patient as the phase of motion. This can be accomplished, for example, using a respiration belt which is worn around the chest of the patient and which measures the movement of the thorax; as an alternative, it is also possible to use position sensors on the chest of the patient in order to record said phase of motion.

It is useful if the simultaneous display of the 3D reconstructed image and the superimposed tip of the catheter on the monitor can be changed by the user, i.e., if it can be rotated or scaled up or down. To make the tip of the catheter more readily recognizable, it can also be displayed in color or it can be made to blink.

According to the present invention, the 3D image data set may be a data set that was acquired prior to the operation. This means that the data set can have been acquired at any time prior to the actual intervention. Any 3D image data set, regardless of the acquisition modality, i.e., for example, a CT, MR, or 3D angiographic X-ray image data set, can be used. All of these data sets allow an exact reconstruction of the area of examination, thus making it possible to visualize this area with anatomic accuracy. As an alternative, it is also possible to use an intraoperatively acquired image data set in the form of a 3D angiographic X-ray image data set. In this context, the term “intraoperative” indicates that this data set is acquired during the same time in which the actual intervention is carried out, i.e., when the patient is already lying on the operating table but before the catheter is inserted, which, however, will take place very shortly after the 3D image data set has been acquired.

Other advantages, features, and details of this invention follow from the practical example described below as well as from the drawings.

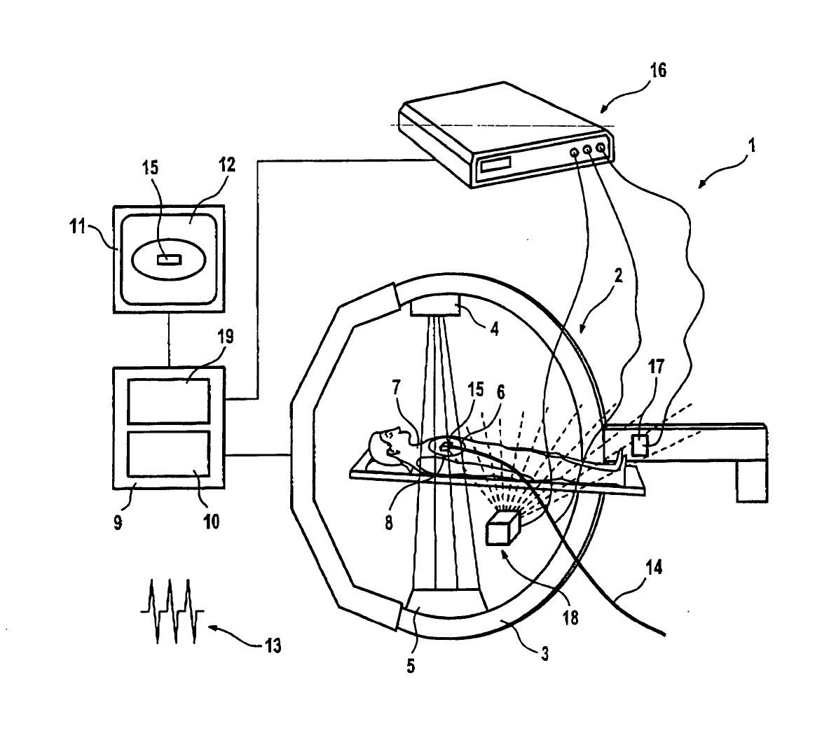

FIG. 1 is schematic sketch of an examination and/or treatment device 1 according to the present invention, in which only the essential components are shown. The device comprises an image-taking device 2 for taking two-dimensional X-ray images. It has a C-shaped arm 3, to which an X-ray radiation source 4 and a radiation detector 5, e.g., a solid state image detector, are attached. The area of examination 6 of patient 7 is located near the isocenter of C-shaped arm 3 so that it is fully visible in the 2D X-ray images. The operation of device 1 is controlled by a control and processing device 9 which, among other things, also controls the image-taking operation. It also comprises an image processing device which is not shown in the drawing.

Using the 2D X-ray images of area of examination 6 which were taken during a rotation of the C-shaped arm by at least 180° around isocenter 8, a 3D image data set 10 is prepared. From this image data set, using 2D X-ray images which show the area of examination—in this case the heart—during the same phase of motion, a 3D reconstructed image of the area of examination is subsequently prepared and displayed on monitor 11. In the example shown, the monitor displays this 3D reconstructed image 12. To ensure that only those images which actually show the heart in the same phase of motion, an ECG 13 is recorded parallel to the acquisition of the 2D X-ray images, as indicated by the tracing shown in the FIGURE. This means that for each 2D X-ray image taken, the associated ECG phase is known so that for the reconstruction, X-ray images in that very same phase can be selected and used.

In the example shown, the 2D X-ray images and thus the 3D image data set 10 are taken immediately prior to the insertion of catheter 14 into area of examination 6. Enclosed in the tip of catheter 14 is a position detection means 15 which is a component of a position locating system 16 which also comprises a position locating sensor 17 that is attached to C-shaped arm 3. As a result, using a suitable calibration routine, the geometric position of isocenter 8 relative to transmitter 18 of the position locating system 16 is known.

Using position locating system 16, it is now possible to determine the spatial position of the tip of the catheter by location position locating means 15 in the coordinate system of position locating system 16. Since, as a result of the chosen configuration of the position locating system and sensor 17 on the C-shaped arm, the coordinate system of the position locating system and the coordinate system of the image-taking device—with which the 2D X-ray images and thus the 3D image data set were taken and in which the 3D reconstructed image 12 was reconstructed—are registered, it is possible to visualize in the 3D reconstructed image 12 the position of the position locating means 15 and thus the tip of the catheter on the basis of the provided coordinates 19 which are entered into the control and processing device 9.

To ensure that the coordinates are recorded in the phase of motion with respect to which the 3D reconstructed image 12 was reconstructed, the recording of the coordinates of the position locating means 15 was here again triggered by ECG 13. Because of the preceding 3D reconstructed image generation, the phase of motion during which this is to take place is known so that the coordinates can be easily recorded in the same phase.

3D reconstructed image 12 can be generated and displayed on the monitor using any modality desired. For example, it can be generated and displayed in the form of an MIP image (MIP=Maximum Intensity Projection) in which the thickness of the image can be interactively modified. Another possibility is the visualization in the form of a VRT image (VRT=Volume Rendering Technique); again, the volume can be interactively clipped. Another possibility is the so-called “fly through” visualization where the viewer is, so-to-speak, inside the tip of the catheter and his direction of sight is determined by the orientation of the tip of the catheter. Those skilled in the art are familiar with all the possibilities of generating the 3D reconstructed image and for visualization it appropriately and can make use of them. In addition, it is, of course, also possible for the user to vary the image in any way imaginable, for example, by rotating or scaling it up, etc. In addition, it is possible to display the tip of the catheter in color.

The position locating system provides five or six degrees of freedom; first, three position parameters (x, y, z) and two orientation parameters (two angles of the tip of the catheter, such as the Euler angle) and optionally the torsion of the catheter (“rolling angle”). This makes it possible to determine both the position and the orientation of the tip of the catheter, into which the position locating means 15 is integrated, in space or in the area of examination and to visualize it in the correct position and orientation on-line in the 3D reconstructed image during the intervention.

1. A method of locating and visualizing a medical catheter that has been introduced into an area of examination of a patient, in particular during a cardiac examination or treatment, comprising the following steps:
using a 3D image data set of the area of examination and generating a 3D reconstructed image of the area of examination,
continuously or intermittently locating the spatial position of the tip of the catheter by means of a position locating system, with a position locating means being integrated into the tip of said catheter,
displaying the 3D reconstructed image and displaying the tip of the catheter in its accurate position in the 3D reconstructed image on a monitor, with the coordinate system of the position locating system and the coordinate system of the 3D reconstructed image being registered with respect to each other.

2. The method as claimed in claim 1 in which defined markings are used to register the coordinate systems in the 3D reconstructed image and, correspondingly, in the coordinate system of the position locating system.

3. The method as claimed in claim 2 in which the markings in the 3D reconstructed image are interactively defined by the user.

4. The method as claimed in claim 2 or 3 in which the markings in the coordinate system of the position locating system are defined by moving the catheter to the marking positions.

5. The method as claimed in claim 4 in which the positions are defined under radiological guidance with the catheter that has been introduced into the area of examination.

6. The method as claimed in claim 2 in which the markings in the 3D reconstructed image are visible markings that have been placed on the outside of the patient.

7. The method as claimed in any one of claims 2 through 6 in which in each coordinate system at least three markings and preferably always the same number of markings are defined.

8. The method as claimed in claim 1 in which, using a segmentation algorithm, several points of the area of examination shown in the 3D reconstructed image are defined and their coordinates are recorded and in which the catheter which has been introduced into the area of examination is moved to a number of points, thus defining them and recording their coordinates, so that each time a specific area is defined by means of the points, and in which for image registration, the transformation matrix is calculated on the basis of the points using a suitable surface matching algorithm.

9. The method as claimed in claim 1 in which a sensor element of the position locating system that is attached to a C-shaped arm of an image-taking device which has an isocenter and with which the 3D image data set has been taken is used, with the 3D reconstructed image being reconstructed relative to the isocenter.

10. The method as claimed in any one of the preceding claims in which, to reconstruct the 3D reconstructed image in a rhythmically or arrhythmically moving area of examination, only those image data are used which are also used during a specific phase of motion that is recorded parallel to the acquisition of the image data, whereby the phase of motion is also recorded while the position of the catheter is being located and the position data are acquired only when the area of examination is in the same phase of motion in which the 3D reconstructed image has been reconstructed.

11. The method as claimed in claim 10 in which, to reconstruct the 3D reconstructed image, only those image data are used which are additionally recorded at a specific time within the phase of motion, with the time also being recorded at the same time that the position of the catheter is being located and with the position data being recorded at the same time during a phase of motion in which the 3D reconstructed image has been reconstructed.

12. The method as claimed in any one of the preceding claims in which the simultaneous display of the 3D reconstructed image and the superimposed tip of the catheter on the monitor can be changed by the user, in particular, can be rotated or scaled up or down.

13. The method as claimed in any one of the preceding claims in which the tip of the catheter in the 3D reconstructed image is blinking or is displayed in color.

14. The method as claimed in any one of the preceding claims in which a preoperatively acquired data set or an intraoperatively acquired data set is used as the 3D image data set.

15. A medical examination and/or treatment device comprising a position locating system, designed to carry out the method as claimed in any one of claims 1 through 14.
